(19) United States
(12) Patent Application Publication
(10) Pub. No.: US 2025/0149112 A1
(43) Pub. Date: May 8, 2025

(54) METHOD FOR DISTINGUISHING STRUCTURAL ISOMERS OF GLYCANS BY SUBSTITUTING SIMILAR MASS ISOTOPES THROUGH COMPUTER SIMULATION

(71) Applicant: TSINGHUA UNIVERSITY, Beijing (CN)

(72) Inventor: Yiying ZHU, Beijing (CN)

(21) Appl. No.: 19/014,253

(22) Filed: Jan. 9, 2025

Related U.S. Application Data
(63) Continuation of application No. PCT/CN2023/125412, filed on Oct. 19, 2023.

(30) Foreign Application Priority Data
Oct. 21, 2022 (CN) 202211293141.8

(51) Int. Cl. G16B 15/20; G16B 40/10; G16C 20/30

(52) U.S. Cl. CPC G16B 15/20; G16B 40/10; G16C 20/30

(57) ABSTRACT

The present application presents a method for distinguishing glycan structural isomers by simulating the replacement of isotopes with similar masses. In this method, an isotope within a structural isomer of a targeted glycan isomer is substituted with another isotope of a similar mass using computer simulations. This process results in a simulated glycan isomer with a slightly altered chemical formula and mass, with the mass difference being less than 0.2 Da). These simulated structural isomers can be quantified based on mass spectrometry data.

### CROSS-REFERENCE TO RELATED APPLICATIONS

This application is a continuation of International Application No. PCT/CN2023/125412, filed on Oct. 19, 2023, which claims priority to Chinese Patent Application No. 202211293141.8, filed on Oct. 21, 2022. The disclosures of the aforementioned applications are hereby incorporated by reference in their entireties.

### FIELD

The present disclosure relates to the field of biotechnology and, particularly, to a method for distinguishing glycan structural isomers by substituting isotopes of elements having similar masses through computer simulation.

### BACKGROUND

Protein glycosylation is a common type of post-translational modification. It is estimated that approximately 50% to 70% of human proteins undergo glycosylation, including surface receptors, organelle-resident proteins, secretory proteins, and transport proteins. This modification plays a crucial role in various biological processes, such as facilitating cell attachment, monitoring protein folding status, enhancing protein delivery, stimulating signal transduction pathways, influencing protein-protein interactions, and modifying protein solubility. Glycans are composed of basic structural units, known as monosaccharides. A glycosidic bond can form between the intramolecular hemiacetal group of one monosaccharide and the hydroxyl group of another. Glucose (Glu/Glc), galactose (Gal), and mannose (Man) are stereoisomers are stereoisomers classified as hexoses (Hex). Deoxyhexose (dHex) is a type of hexose in which a hydroxyl group is substituted by a hydrogen atom, such as fucose (Fuc). (N-acetylglucosamine (GlcNAc) and N-acetylgalactosamine (GalNAc) are both classified as N-acetylhexosamine (HexNAc). Sialic acid is a general term for substituted neuraminic acids that contains nine carbon atoms, with N-acetylneuraminic acid (NeuAc) and N-glycolylneuraminic acid (NeuGc) being common in mammals. NeuAc is widely present in human proteins, while NeuGc is a non-human sialic acid that has been found in apes. Glycosylation can be categorized into several subtypes based on the different types of glycosidic bonds: N-linked glycosylation, O-linked glycosylation, C-linked glycosylation, and phosphoglycosylation. The most common forms are N-linked glycans (N-glycan database) associated with N-linked glycosylation and O-linked glycans (O-glycan database) associated with O-linked glycosylation. N-linked oligosaccharides are bonded to the nitrogen atom of asparagine (Asn), while O-linked glycosylation involves attaching glycans to the oxygen atom in serine, threonine, or tyrosine. N-linked glycans share a common pentasaccharide core structure and can generally be divided into three different subtypes: high mannose, complex, and hybrid.

Due to the vast variety and complex structures of glycans, mass spectrometry analysis of glycosylation is more challenging than that of other post-translational modifications of proteins. There are generally two categories of methods for analyzing protein glycosylation. The first category involves releasing glycans from proteins through enzymatic hydrolysis, followed by the specific analysis of pure sugar molecules or peptides. The second category focuses on the direct analysis of glycopeptides, which contain information about the glycosylation sites. The presence of structural isomers, which can have the same parent ion mass in a mass spectrum due to different glycan branch linkages, further complicates the analysis process. However, advancements in mass spectrometry technology, including the development of secondary and even multi-stage mass spectra, allow for further dissociation of glycan molecules, enabling the analysis of these structural isomers. In recent years, software tools for large-scale searches of glycan molecules and glycopeptides have been developed, such as pGlyco, ProteinProspector, and O-Pair. The field of glycoproteomics has progressed from qualitative to quantitative analysis. This means not only to identifying the types of glycosylation present in different groups of proteins but also quantifying the various glycans. Large-scale quantitative methods for molecules identified by mass spectrometry have been continually updated and optimized. For instance, peptide quantification methods have evolved from spectral counting (using the number of secondary spectra) to the use of primary spectra (MS1) peak area, as well as techniques like iTRAQ and TMT, and to the application of heavy isotope-labeled peptides as standards for precise quantification, prompting the development of corresponding analysis software. Data-dependent analysis (DDA) is the most commonly used scanning mode in mass spectrometry. This method involves selecting the highest abundance ions for fragmentation and then scanning the MS/MS spectra. Following this, relative quantification is achieved by using the peak area or peak height of MS1 ions from various samples. However, due to the principles of DDA scanning, some peptides may not be selected for secondary MS/MS analysis in certain samples, leading to missing values. Consequently, even if the peptides are present, they may lack a secondary spectrum and quantitative data in the final results. To address this issue, specialized quantitative software such as Progenesis and Skyline has been developed. For example, the Match-between-runs algorithm allows for peak extraction based on other ion characteristics, such as retention time, which can significantly reduce missing values and enhance repeatability. Many software programs even include visualization windows for manual adjustments. However, when it comes to glycopeptides, there can be challenges in utilizing quantitative software like Skyline, which is primarily designed for standard peptide molecules with fixed mass protein modifications and not specifically tailored for complex glycan modifications. The software requires input that includes the identified peptide sequence and modification mass. Since it only uses the modification mass as an identifier, different glycopeptide-linked glycan isomers may be incorrectly classified as the same peptide.

### SUMMARY

In a first aspect, the present disclosure provides a method for quantitatively analyzing glycan isomers based on mass spectrometry data. The method includes: substituting, by means of computer simulation, an isotope in a structural isomer of a to-be-quantified glycan isomer with an isotope having a similar mass, to obtain a simulated glycan isomer with a changed chemical formula and mass; and quantifying the simulated glycan isomer based on the mass spectrometry data, to obtain quantitative results of different structural isomers. A difference between a mass of the simulated glycan isomer and a mass of the to-be-quantified glycan isomer is less than or equal to 0.2 Da.

In a second aspect, the present disclosure provides a method for quantitatively analyzing a glycopeptide containing glycan isomers based on mass spectrometry data. The method includes: substituting, by means of computer simulation, an isotope in the glycan isomer contained in the glycopeptide with an isotope having a similar mass, to obtain a simulated glycan isomer with a changed chemical formula and mass, and to obtain a glycopeptide containing the simulated glycan isomer; and quantifying, by means of a mass spectrometry data quantification software, the glycopeptide containing the simulated glycan isomer based on the mass spectrometry data, to obtain quantitative results of the glycopeptide containing the glycan isomers of different structures. A difference between a mass of the simulated glycan isomer and a mass of the glycan isomer is less than or equal to 0.2 Da.

In a third aspect, the present disclosure provides a device for quantitatively analyzing a glycopeptide containing glycan isomers in mass spectrometry data. The device may include the following modules: B1) mass spectrometry data acquisition module configured to acquire mass spectrometry data of a sample; B2) glycopeptide identification module configured to identify a glycopeptide contained in the sample based on the mass spectrometry data; and B3) glycopeptide quantification module configured to quantify the glycopeptide. The glycopeptide quantification module includes the following modules: B3-1) glycan isomer simulation module configured to obtain a simulated glycan isomer and a glycopeptide containing the simulated glycan isomer through computer simulation of the glycan isomers having different structures contained in the glycopeptide; and B3-2) glycopeptide quantification module configured to quantify, by means of a mass spectrometry data quantification software, the glycopeptide containing the simulated glycan isomer, to obtain quantitative results of the glycopeptide containing the glycan isomer.

In a fourth aspect, the present disclosure further provides a computer-readable storage medium having a computer program stored thereon. The computer program enables a computer to perform steps of the methods described above.

### DETAILED DESCRIPTION

The present disclosure is further described in detail below in conjunction with specific embodiments. The embodiments are only for illustrating the present disclosure, rather than for limiting the scope of the present disclosure. The following embodiments are provided as a guide for further improvements by those skilled in the art and are not intended to limit the present disclosure in any way.

The technical problem to be solved by the present disclosure is how to distinguish a glycan structural isomer based on mass spectral data quantification software and/or how to quantify a glycan structural isomer in mass spectral analysis and/or how to quantify a glycan structural isomer based on the mass spectral data quantification software.

In order to solve the above-mentioned technical problem, the present disclosure first provides a method for quantitatively analyzing glycan isomers based on mass spectrometry data. The method includes: substituting, by means of computer simulation, an isotope in a structural isomer of a to-be-quantified glycan isomer with an isotope having a similar mass, to obtain a simulated glycan isomer with a changed chemical formula and mass; and quantifying the simulated glycan isomer based on the mass spectrometry data, to obtain quantitative results of different structural isomers.

A difference between a mass of the simulated glycan isomer and a mass of the to-be-quantified glycan isomer is less than or equal to 0.2 Da.

The to-be-quantified glycan isomer may be a structural isomer having the same molecular formula but different structural arrangements of atoms.

The isotope having a similar mass may be a combination of isotopes having a mass difference of no more than 0.05 Da. For example, for ¹⁴N, the isotope having a similar mass may be ¹³C and ¹H; for ¹⁶O, the isotope having a similar mass may be ¹⁵N and ¹H; and for ¹⁵N, the isotope having a similar mass may be ¹²C and ¹H.

In the above-mentioned method, x is a serial number of respective structural isomers of the to-be-quantified glycan isomers sorted in ascending order of glycan ID number. The serial number is a natural number from 1 to n. The computer simulation is performed based on the number of N and the number of O in a chemical formula of the to-be-quantified glycan isomer. The computer simulation includes any one of the following steps:
A1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
A2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
A3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, until k ¹⁶O are removed; for the structural isomer with the serial number of x, removing m ¹⁴N from the chemical formula, adding m ¹³C and m ¹H in the chemical formula, and removing (x−m−1) ¹⁶O from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da.

The to-be-quantified glycan isomer may include n structural isomers, where n is a natural number.

The number of N in the chemical formula is m, where m is a natural number.

The number of O in the chemical formula is k, where k is a natural number.

x may be the serial number of respective structural isomers of the to-be-quantified glycan isomers sorted in an ascending order of glycan ID number. The serial number is a natural number from 1 to n.

The glycan ID number may be derived from GlycomeDB database (related website: www.glycome-db.org).

The mass spectrometry data quantification software may be Skyline software.

In order to solve the above-mentioned technical problem, the present disclosure further provides a method for quantitatively analyzing a glycopeptide containing glycan isomers based on mass spectrometry data. The method includes: substituting, by means of computer simulation, an isotope in the glycan isomer contained in the glycopeptide with an isotope having a similar mass, to obtain a simulated glycan isomer with a changed chemical formula and mass, and to obtain a glycopeptide containing the simulated glycan isomer; and quantifying, by means of a mass spectrometry data quantification software, the glycopeptide containing the simulated glycan isomer based on the mass spectrometry data, to obtain quantitative results of the glycopeptide containing the glycan isomers of different structures.

A difference between a mass of the simulated glycan isomer and a mass of the glycan isomer is less than or equal to 0.2 Da.

The glycan isomer may be an isomer.

The isotope having a similar mass may be a combination of isotopes having a mass difference of no more than 0.05 Da. For example, for ¹⁴N, the isotope having a similar mass may be ¹³C and ¹H; for ¹⁶O, the isotope having a similar mass may be ¹⁵N and ¹H; and for ¹⁵N, the isotope having a similar mass may be ¹²C and ¹H.

In the above-mentioned method, x is a serial number of respective structural isomers of the to-be-quantified glycan isomers sorted in ascending order of glycan ID number. The serial number is a natural number from 1 to n. The computer simulation is performed based on the number of N and the number of O in a chemical formula of the to-be-quantified glycan isomer. The computer simulation includes any one of the following steps:
A1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
A2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
A3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, until k ¹⁶O are removed; for the structural isomer with the serial number of x, removing m ¹⁴N from the chemical formula, adding m ¹³C and m ¹H in the chemical formula, and removing (x−m−1) ¹⁶O from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da.

x is the serial number of respective structural isomers of the glycan isomers sorted in ascending order of glycan ID number. The serial number is a natural number from 1 to n. The glycan ID number may be derived from the GlycomeDB database (related website: www.glycome-db.org).

In the above-mentioned method, the mass spectrometry data quantification software may be Skyline software.

In order to solve the above-mentioned technical problem, the present disclosure further provides a device for quantitatively analyzing a glycopeptide containing glycan isomers in mass spectrometry data. The device may include the following modules: B1) mass spectrometry data acquisition module configured to acquire mass spectrometry data of a sample; B2) glycopeptide identification module configured to identify a glycopeptide contained in the sample based on the mass spectrometry data; and B3) glycopeptide quantification module configured to quantify the glycopeptide. The glycopeptide quantification module includes the following modules: B3-1) glycan isomer simulation module configured to obtain a simulated glycan isomer and a glycopeptide containing the simulated glycan isomer through computer simulation of the glycan isomers having different structures contained in the glycopeptide; and B3-2) glycopeptide quantification module configured to quantify, by means of a mass spectrometry data quantification software, the glycopeptide containing the simulated glycan isomer, to obtain quantitative results of the glycopeptide containing the glycan isomer.

In the above-mentioned device, x is a serial number of respective structural isomers of the to-be-quantified glycan isomers sorted in ascending order of glycan ID number, the serial number being a natural number and ranging from 1 to n. The computer simulation is performed based on the number of N and the number of O in a chemical formula of the to-be-quantified glycan isomer. The computer simulation includes any one of the following steps:
C1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
C2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
C3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, until k ¹⁶O are removed; then for the structural isomer with the serial number of x, removing m ¹⁴N from the chemical formula, adding m ¹³C and m ¹H in the chemical formula, and removing (x−m−1) ¹⁶O from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da.

The glycan isomer may include n structural isomers, where n is a natural number.

m is a natural number. k is a natural number.

The serial number is a natural number from 1 to n.

The glycan ID number is derived from the GlycomeDB database (related website: www.glycome-db.org).

In the above-mentioned device, the mass spectrometry data quantification software may be Skyline software.

In order to solve the above-mentioned technical problem, the present disclosure further provides a computer-readable storage medium having a computer program stored thereon. The computer program enables a computer to perform steps of the methods as described above.

According to the present disclosure, glycopeptides containing sialic acid in the serum of the liver cancer patient and normal human serum were analyzed based on mass spectrum, and a total of 1,218 glycopeptides were identified by searching with pGlyco software. By using the method of distinguishing glycan structural isomers by substituting isotopes having a similar mass through computer simulation established in the present disclosure, the glycan isomers of the 1,218 glycopeptides were distinguished by finely adjusting the mass of the glycan isomers, and all the identified glycopeptides were quantified using Skyline software. The results indicate that there were no missing values for glycopeptides, and it was finally found that the changes of 315 glycopeptides in the serum of liver cancer patients were greater than 2.5 times the changes of those in the normal human serum. The experiments demonstrate that the method established in the present disclosure can effectively distinguish different glycopeptide-linked glycan isomers, while accurately performing quantitative and differential analysis on the identified glycopeptides without missing values

Compared with the related art, the present disclosure has the following beneficial effects.

In the present disclosure, glycan structural isomers are distinguished by in silico substituting isotopes having a similar mass, enabling the software to distinguish isomers and perform separate quantification.

In the present disclosure, glycan structural isomers are distinguished by in silico substituting isotopes having a similar mass adopting in silico, enabling the mass spectrometry data quantitative software to distinguish isomers and quantify the isomer molecules separately.

The experimental methods in the following embodiments are all conventional methods unless otherwise specified. Unless otherwise specified, the materials, reagents, instruments, etc. used in the following examples are commercially available. The quantitative tests in the following examples were all repeated twice, and the results were averaged.

The sources of reagents or consumables in the examples of the present disclosure were as follows:
4-hydroxyethyl piperazine ethane sulfonic acid: Sigma-Aldrich 54457;
Pierce BCA kit: ThermoFisher 23227;
Dithiothreitol: Invitrogen 15508013;
Iodoacetamide: Sigma-Aldrich H4034;
Pancreatin: Promega V5113;
Formic acid: Fisher A117-50;
Solid phase extraction C18 column: CDS 4215SD;
IMAC Fe-NTA: ThermoFisher A32992; and
C18 stagetip: CDS Empore 2215.

### Example 1: Establishment of a Method for Distinguishing a Glycan Structural Isomer by Substituting an Isotope Having a Similar Mass Through Computer Simulation

### 1. Sample Collection

One patient diagnosed with liver cancer at Shandong Provincial Hospital and one healthy person were selected as subjects for sample collection. The study protocol was approved by the Ethics Committee of Shandong Provincial Hospital, and the study was conducted according to the principles of the Declaration of Helsinki. Before enrollment, each participant or his/her legal representative signed a written informed consent.

Whole blood was collected from the liver cancer patient and the healthy subject, and serum samples from the liver cancer patient and the healthy subject were obtained by centrifugation.

### 2. Mass Spectrometry Identification and Quantification of Sialic Acid N-Glycan Peptide

### 2.1 Sample Preparation and Mass Spectrometry Detection

### 2.1.1 Sample Preparation

### 2.1.1.1 Serum Proteolysis

Serum samples from the liver cancer patient and the healthy subject were dissolved in 4× volume lysis buffer (solution composition: 9 M urea and 20 mM 4-hydroxyethyl piperazine ethane sulfonic acid) and centrifuged at 16,000×g for 5 minutes. The supernatant was collected as the dissolved serum protein solution. The protein concentration in the dissolved serum protein solution from each of the two samples was determined using the Pierce BCA kit.

Thereafter, 1 mg of dissolved serum protein solution was taken and added with dithiothreitol to a final concentration of 4.5 mM, and the mixture reacted at room temperature for 1 hour. Then, iodoacetamide was added to a final concentration of 10 mM, and the mixture reacted at room temperature for half an hour in the dark. Pancreatin was added according to a mass ratio of enzyme:protein=1:20 (w:w), and the mixture reacted at room temperature overnight, to obtain a serum protease hydrolysate. Formic acid was added to the serum protease hydrolysate to a final concentration of 0.1%, and a purified serum protease hydrolysate was obtained by desalting using the solid phase extraction C18 column for later use.

### 2.1.1.2 Enrichment of Sialic Acid Glycopeptide

The glycopeptide containing sialic acid was enriched using Fe-NTA IMAC beads. According to the experimental steps in the kit manual, 0.5 mg of purified serum protease hydrolysate was taken and mixed with IMAC beads for one hour. After elution, the serum protease hydrolysate was spin-dried and resuspended in 0.1% formic acid solution. It was then desalted with the C18 stagetip, spin-dried, and redissolved in 50 μL of 0.1% formic acid.

### 2.1.2 Mass Spectrometry Detection

LC-MS/MS: Thermo Fisher U3000 nanoUPLC was used together with a Thermo Fisher 3-in-1 tandem Orbitrap Eclipse mass spectrometer for detection. 50 cm (100 μm ID, 1.9 μm C18 packing) analytical column was used. In the liquid phase, solution A was an aqueous solution of 0.1% formic acid, and solution B was an aqueous solution of 80% acetonitrile and 0.1% formic acid. The injection volume was 4 μL, and the detection was repeated twice for each sample. The liquid phase gradient increased from 4% to 50% in 90 minutes. The composition of solvent B was 80% acetonitrile and 0.1% formic acid aqueous solution, and the flow rate of solvent B was 0.3 μL/min.

Both primary mass spectrometry data and secondary mass spectrometry data were acquired with an orbitrap mass analyzer with high mass accuracy and high sensitivity: primary scan range (m/z)=800 to 2,000; resolution=120,000; AGC=200,000; maximum injection time=100 ms; included charge state=2 to 6; dynamic exclusion after n times, n=1; dynamic exclusion duration=15 s; mass spectrometry fragmentation mode set to stepped HCD (NCE=30%+10%); secondary isolation window=2; resolution=15,000; AGC target=500,000; maximum injection time=250 ms. After mass spectrometry scanning, a raw file was generated. The raw file corresponding to the sample of the liver cancer patient was named Cancer. raw, while the raw file corresponding to the sample of the healthy subject was named Normal. raw.

### 2.2 Glycopeptide Identification:

Default search parameters of pGlyco 2.0 software (download website: http://pfind.org/software/pGlyco/index.html) were used. The UniProt human protein sequence database and the human N-linked glycan database (N-glycan database) used by pGlyco in 2020 were selected, containing a total of 8093 glycan IDs, and the Total FDR was set to 1%. The searched glycopeptide identification data were in txt files, named Cancer.txt (corresponding to the liver cancer patient) and Normal.txt (corresponding to the healthy subject).

### 2.3 Glycopeptide Quantification

### 2.3.1 Format Conversion of Glycan Database

The glycan database (N-glycan database) was converted into a format acceptable to mass spectrometry data peptide quantification software Skyline (download website: https://skyline.ms/project/home/software/skyline/begin.view).

### 2.3.1.1 Format Conversion of Glycan

In the identification results of the glycan database (N-glycan database) obtained in step 2.2, Glycan ID 127 was used as an example to describe the format conversion. In the original Glycan database, the parameter of Glycan ID 127 was “kind=43100”, having the meanings of Hex-4, HexNAc=3, NeuAc=1, NeuGc=0, and Fuc=0, and the chemical formula of Glycan ID 127 was C59H96N4043. In the converted new format, the parameter of Glycan ID 127 was <static_modification, aminoacid=“N”, explicit_decl=“true”, formula=“C59H96N4043”, name=“127”/>.

All glycans, including both non-isomeric and isomeric glycans, were subjected to a format conversion, and the resulting file was saved as “regular glycans.txt”.

### 2.3.1.2 Format Conversion of Glycan Isomer

The masses of glycan isomers with the same chemical formula and mass were slightly adjusted; that is, isotopes in the glycan isomers were substituted with isotopes having similar masses through computer simulation to obtain simulated glycan isomers with a changed chemical formula and mass. After changing the chemical formula of the glycan isomer, the mass (molecular weight) of the glycan isomer was changed slightly. Based on such a slight change, the glycan isomers were distinguished in the subsequent analysis software, and the original glycan isomer was determined according to this rule at the end of the analysis. In the final result output, the original chemical formula and structure of the original glycan isomer were output. The specific steps were as follows:

All glycan isomers (n glycan isomers) with the same mass were found and sorted in ascending order of glycan ID number. The serial number thereof was recorded as x (x is a natural number ranging from 1 to n). The chemical formula and mass of the glycan isomers were slightly adjusted and changed (with a mass change of less than 0.2 Da) using the computer simulation (in silico) according to the following rules.
I. When the number (m) of N in the chemical formula is greater than or equal to the number of the structural isomers of the glycan isomers minus 1 (i.e., n−1), for an isomer with the serial number of x, removing (x−1) ¹⁴N from the chemical formula, and adding (x−1) ¹³C and (x−1) ¹H in the chemical formula. Compared with the original glycan isomer, the mass is increased by (x−1)×0.008106 Da.
II. When the number (m) of N in the chemical formula is smaller than the number of the structural isomers of the glycan isomers minus 1 (i.e., n−1), and when a sum (m+k) of the number (m) of N and the number (k) of O is greater than or equal to the number of the structural isomers minus 1 (i.e., n−1), for the isomer with the serial number of x, removing (x−1) ¹⁴N from the chemical formula, and adding (x−1) ¹³C and (x−1) ¹H in the chemical formula, until m ¹⁴N are removed; then, for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, to obtain the simulated glycan isomer. Compared with the to-be-quantified glycan isomer, the mass of the simulated glycan isomer is increased by (x−1)×0.008106 Da or m×0.008106 Da+(x−m−1)×0.013019 Da.
III. When the sum (m+k) of the number (m) of N and the number (k) of O in the chemical formula is smaller than the number of the structural isomers of the glycan isomers minus 1 (i.e., n−1), for the isomer with the serial number of x, removing (x−1) ¹⁴N from the chemical formula, and adding (x−1) ¹³C and (x−1) ¹H in the chemical formula, until m ¹⁴N are removed; then, for the structural isomer with the serial number of x, removing m ¹⁴N from the chemical formula, adding m ¹³C and m ¹H in the chemical formula, and removing (x−m−1) 160 from the chemical formula, and adding (x−m−1) ¹⁵N and (x−m−1) ¹H in the chemical formula, until k ¹⁶O are removed; then for the structural isomer with the serial number of x, removing m ¹⁴N from the chemical formula, adding m ¹³C and m ¹H in the chemical formula, and removing (x−m−1) ¹⁶O from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer. Compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da.

For example, as shown in Table 1, it was identified that the human serum glycopeptide contains 6 glycan isomers (n=6) with the same chemical formula C90H146N6O65 and the same mass 2350.83035 Da, which were modified to have different structures (having a sufficient amount of N in the chemical formula: the number of N (m=6) is greater than the number of structural isomers minus 1 (i.e., m>n−1=5)), with the glycan ID from 1266 to 1273 (ID serial number from 1 to 6), respectively. Through the computer simulation, the masses of glycan isomers changed slightly after changing the chemical formula:
for the glycan with ID serial number x of 1 (glycan ID 1266): the chemical formula and mass were not simulated and did not change;
for the glycan with ID serial number x of 2 (glycan ID 1267): through the computer simulation, the chemical formula was changed from C90H146N6O65 to C90H147N5O65C′1, that is, (2-1) (i.e., one) ¹⁴N was removed from the chemical formula, and (2-1) (i.e., one) ¹³C and one ¹H were added; compared with the mass of the original glycan isomer, the mass was increased by (2-1)×0.008106 Da;
for the glycan with ID serial number x of 3 (glycan ID 1269): through the computer simulation, the chemical formula was changed from C90H146N6O65 to C90H148N4O65C′2, that is, (3-1) (i.e., two) ¹⁴N was removed from the chemical formula, (3-1) (i.e., two) ¹³C and two ¹H were added; compared with the mass of the original glycan isomer, the mass was increased by (3-1)×0.008106 Da;
for the glycan with ID serial number x of 4 (glycan ID 1270): through the computer simulation, the chemical formula was changed from C90H146N6O65 to C90H149N3O65C′3, that is, (4-1) (i.e., three) ¹⁴N was removed from the chemical formula, and (4-1) (i.e., three) ¹³C and three ¹H were added; compared with the mass of the original glycan isomer, the mass was increased by (4-1)×0.008106 Da;
for the glycan with ID serial number x of 5 (glycan ID 1272): through the computer simulation, the chemical formula was changed from C90H146N6O65 to C90H150N2O65C′4, i.e., (5-1) (i.e., four) ¹⁴N was removed from the chemical formula, and (5-1) (i.e., four) ¹³C and four ¹H were added; compared with the mass of the original glycan isomer, the mass was increased by (5-1)×0.008106 Da; and
for the glycan with ID serial number x of 6 (glycan ID 1273): through the computer simulation, the chemical formula was changed from C90H146N6O65 to C90H151N1O65C′5, that is, (6-1) (i.e., five) ¹⁴N was removed from the chemical formula, and (6-1) (i.e., five) ¹³C and five ¹H were added; compared with the mass of the original glycan isomer, the mass was increased by (6-1)×0.008106 Da.

According to the masses of the glycan isomers obtained after simulation, the glycan isomers may be distinguished in subsequent analysis software.

TABLE 1
Comparison of original glycan isomer before and after fine-tuning
of chemical formula through computer simulation

| Original glycan isomer | | | | Glycan isomer changed by | |
|---|---|---|---|---|---|
| Glycan | Chemical | | | in silico simulation | |
| ID | formula | Structure | Mass (Da) | Chemical formula | Mass (Da) |
| 1266 | C90H146N6O65 | (N(F)(N(H(H)(H(N(H(A)))(N(H(A))))))) | 2350.83035 | C90H146N6O65 | 2350.83035 |
| 1267 | C90H146N6O65 | (N(N(H(H)(H(N(H(A)))(N(F)(H(A))))))) | 2350.83035 | C90H147N5O65C′1 | 2350.83846 |
| 1269 | C90H146N6O65 | (N(F)(N(H(N)(H(H))(H(N(H(A(A))))))) | 2350.83035 | C90H148N4O65C′2 | 2350.84656 |
| 1270 | C90H146N6O65 | (N(F)(N(H(H(N(H(A))))(H(N(H(A))))))) | 2350.83035 | C90H149N3O65C′3 | 2350.85467 |
| 1272 | C90H146N6O65 | (N(N(H(N)(H(H))(H(N(F)(H(A(A)))))))) | 2350.83035 | C90H150N2O65C′4 | 2350.86277 |
| 1273 | C90H146N6O65 | (N(N(H(H(N(H(A))))(H(N(F)(H(A))))))) | 2350.83035 | C90H151N1O65C′5 | 2350.87088 |

Note:
C′ represents a heavy isotope labeled ¹³C.

After all glycan isomers in the glycan database were converted through the computer simulation, the resulting file was saved as a new database named shifted glycans.txt.

### 2.3.2 Format Conversion of Glycopeptide

Glycopeptides were searched and identified in the pGlyco database, and the glycopeptide result file (txt format) obtained by the search was converted into a pepXML file. The parameter settings for each glycopeptide in the specific pepXML file were as follows:
a. The searched glycopeptide result file information such as file name, search software, and the like was extracted and placed at the beginning of the pepXML file, such as: </analysis_summary>, <msms_run_summary, base_name=“Cancer”, raw_data=“.raw”, raw_data_type=“.raw”>, <fragment_mass_type=“monoisotopic”, precursor_mass_type=“monoisotopic”, search_engine-“pGlyco”>, where: analysis_summary represents the analysis summary; msms_run_summary represents the secondary mass spectrometry analysis summary; base_name represents the base name; raw_data represents the raw data name; raw_data_type represents the raw data type; fragment_mass_type represents the fragmentation mass type; precursor_mass_type represents the precursor ion mass type; and search_engine represents the search software.
b. The modification was defined in the section of <analysis_summary>aminoacid_modification, and all modifications in this file were found in the pGlyco glycopeptide search results, and then converted to the format <aminoacid_modification, aminoacid=“X”, description=“XX”, mass=“XX.XXXXXXXX”, massdiff=“XX.XXXXXXXX”, variable=“Y/N”/>, where: aminoacid_modification represents amino acid modification; aminoacid represents the modified amino acid; Massdiff represents the mass of the modification group; mass represents the mass of the massdiff+amino acid residue; variable represents variable modification; and description represents the functional description.

Definitions of all common modifications were as follows, for example, <aminoacid_modification, aminoacid=“C”, massdiff=“57.02146374”, mass=“160.030648219”, variable=“N”, description=“Carboaminomethyl”/>.

Definitions of all glacan moieties were as follows, for example, <aminoacid_modification, aminoacid=“N”, description=“GlycanID1270”, mass=“2464.873277”, massdiff=“2350.83035”, variable=“Y”/>.
c. The searched results of glycopeptides were filled in the section of search summary according to the mass spectrometry (MS/MS) spectrum number, i.e., scan No., including assumed_charge (assumed charge), precursor_neutral_mass (precursor neutral mass), scan, probability, calc_neutral pep_mass (calculated mass of the obtained neutral peptide), protein info (information of protein), etc.

For example:

```
  </search summary>
  <spectrum_query , assumed_charge=“4” , end_scan=“60244” , index=“1” ,
precursor_neutral_mass=“3744.68906” , spectrum=“Cancer.60244.60244.4” ,
start_scan=“60244”> , <search_result , search_id=“1”> , <search_hit
calc_neutral_pep_mass=“3744.6715200000003” , hit_rank=“1” , is_rejected=“0” ,
massdiff=“−0.0000602” , peptide=“VDKDLQSLEDILHQVENK” ,
protein=“sp|P02679|FIBG_HUMAN”> , <analysis_result , analysis=“peptideprophet”> ,
<peptideprophet_result probability=“0.1287470509485”/>
  </analysis_result>
  <modification_info>
    <mod_aminoacid_mass mass=“1736.624537” position=“17”/>
    </modification_info>
    </search_hit>
    </search_result>
    </spectrum_query>
```

For the modification section, column about modification was found in the glycopeptide results obtained from the pGlyco search, and the modification position and specific modification were found. They were converted into the format of pepXML file, that is, mod_aminoacid_massposition (sequential position of amino acids on the peptide segment)=“X”, and mass (mass of modified amino acids)=“>XXXX.XXXXXXXX”. For example, the glycopeptide search result “1, Carbamidomethyl [C]” was converted into a pepXML file as <mod_aminoacid_massposition=“1”, mass=“160.030648219”/>.

The glycan modifications were added according to the glycan modification masses in the regular glycans.txt file or the shifted glycans.txt file obtained in step 2.3.1.
d. The names of the two converted files (corresponding converted results of the data of the liver cancer patient or the healthy subject, Cancer or Normal) were changed to be the same as the glycopeptide result files (Cancer or Normal) obtained by the database search, and the file extension was changed as pep.xml. They were labeled in different folders.

### 2.3.3 Format Conversion of Mass Spectrometry Scan File

The original raw file (with the name of Cancer. raw or Normal. raw) obtained in step 2.1.2 was converted into mzXML format using MSconvert software (download website: https://proteowizard.sourceforge.io/).

### 2.3.4 Creation of Excel Report of Total Results

A template was established to convert the pGlyco glycopeptide identification results into the final set of glycopeptide qualitative and quantitative results. The report included the report Cancer.txt/Normal.txt of pGlyco glycopeptide identification in step 2.2 and shifted glycans.txt of glycan mass converted in step 2.3.1, Gene name, Protein name, Accession protein number in the database, kD protein mass, Site glycan modification site, GlyID glycan number, Glycan glycan composition, Glymass normal glycan mass, Calc.m/z theoretical glycopeptide charge-to-mass ratio, PlausibleStruct possible glycan structure, Peptide glycan-modified peptide sequence, Charge number, GlycoPeptide modified peptide sequence (changing the modified aspartic acid originally substituted by J in the pGlyco search result back to N, and adding the normal modified mass [+XXXX.XXX] after the modified amino acid, and such a modified peptide format may be accepted by Skyline), Shift GlycoPeptide (same as GlycoPeptide, but according to the rule of shifted glycans, the modified glycan was substituted with the changed mass), PPM (glycopeptide mass change), Total area (Cancer, Normal) the peak area of glycopeptides included controls between the liver cancer patient and the healthy subject (this item was reserved for the next step 2.3.5, leave it blank for now).

### 2.3.5 Quantification of Glycopeptide

The quantification of glycopeptide was performed using Skyline (MacCoss Lab) software. The specific steps were as follows:
a. A skyline project was created, and the file name was saved as test. Three skyline files with expanded names of sky, sky. view, and skyd were found. The test. sky file was right-clicked and opened with Notepad. The “regular glycans.txt” file obtained in step 2.3.1.1 was opened, and the complete glycan chain mass list thereof was copied and inserted into the parameter section of static_modification. This step was the import of the modification definition.
b. The parameters of Skyline were set as follows:

Peptide Settings: Digestion Enzyme, Trypsin; Ion Transition Setting: Precursor Charge 2, 3, 4, 5, 6, 7, Ion Charges 1, 2, 3, 4, 5, 6, Ion Types y, b, p; resolving power (matching mass spectrum MS1 settings): Resolving Power 120,000 at 200 m/z.
c. The Skyline file test was saved, and the file test. sky was double-clicked. The pep.xml file converted in step 2.3.2 was used, and a library was normally created under the Peptide Settings-Library tab of the test. sky file (cutoff score was 0).
d. Back to the report in step 2.3.4, the specific values (ppm) of the mass change of each glycopeptide in the report were listed and sorted in ascending order. The glycopeptide list of normal glycan mass peptides (excluding glycan isomers, ppm=0) was copied and pasted into the left side of the test. sky main interface, making sure all peptides match the library spectrum. The raw file was imported, and the peak value thereof was manually adjusted as usual. The final report was exported.
e. For glycopeptides with changed mass (including glycan isomers and computer-simulated mass changes), the Skyline file was re-created and named shifted test, and steps a to c were repeated (importing the modification definition in step a; and using the “shifted glycans.txt” file obtained in step 2.3.1.2 and the pepXML file with changed mass obtained in step 2.3.2 when building the library in step c). In the report in step 2.3.4, the glycopeptides were divided into two columns: 0 ppm to 10 ppm and 10 ppm to 50 ppm. The mass accuracy was adjusted under the Transition Settings-Full Scan tab, and analyzed separately. It was ensured that all peptides matched the library spectra. The raw file was imported, and the peak values thereof were manually adjusted as usual. Two Skyline glycopeptide peak area reports were exported.
f. Quantitative reports of glycopeptide peak areas for normal mass (ppm=0) and changed mass (0<ppm<10, 10<ppm<50) were combined. The quality change threshold may be adjusted according to the needs of different projects. Finally, the peak area was pasted into the Total area column of the general report in step 2.3.4 by searching the corresponding glycopeptide sequence, and finally, the shifted GlycoPeptide and PPM columns were deleted.

In summary, glycopeptides containing sialic acid in the serum of liver cancer patients were analyzed alongside those from normal human serum using mass spectrometry. A total of 1,218 glycopeptides were identified through a search conducted with pGlyco software. However, the presence of isomers in the glycosyl modifications of these glycopeptides made it difficult to accurately quantify and differentially analyze the identified glycopeptides without encountering missing values. To address this challenge, a method was employed for distinguishing glycan structural isomers by substituting isotopes with similar masses through computer simulation as established in this disclosure. This approach allowed us to finely adjust the mass of the glycan isomers and successfully distinguish them within the glycopeptides. All identified glycopeptides were quantified using Skyline software, resulting in no missing values. Ultimately, our findings revealed that the levels of 315 glycopeptides in the serum of liver cancer patients changed by more than 2.5 times compared to those observed in the normal human serum.

The present disclosure has been described in detail above. It is clear to those skilled in the field that it can be implemented in a broader range using equivalent parameters, concentrations, and conditions, all without deviating from the essence and scope of this disclosure and without requiring unnecessary experiments. While specific embodiments have been present, it should be understood that further improvements can be made. In summary, according to the principles outlined in the present disclosure, this application aims to encompass any changes, uses, or enhancements, including modifications that extend beyond the scope outlined in this application and are made using conventional techniques familiar to those in the field. Certain essential features may be applied within the scope defined by the following claims.

### INDUSTRIAL APPLICATIONS

The disclosure presents an analysis of glycopeptides containing sialic acid found in the serum of liver cancer patients compared to normal human serum, utilizing mass spectrometry. A total of 1,218 glycopeptides were identified through searches conducted with pGlyco software. A novel method was developed to distinguish glycan structural isomers by substituting isotopes with similarly weighted alternatives through computer simulation. This approach enabled precise adjustments to the masses of the glycan isomers, facilitating the differentiation of isomers associated with the identified glycopeptides. All identified glycopeptides were quantified using Skyline software, and the results indicated that there were no missing values for the analyzed glycopeptides. Changes in the serum of liver cancer patients revealed that 315 glycopeptides were observed at levels over 2.5 times greater than those found in normal human serum. The method described in this disclosure allows for the simultaneous quantification and differential analysis of glycopeptides with complex modifications of various glycans, achieving accurate results without missing values. This method can be utilized for the development of products that distinguish different glycopeptide-linked glycan isomers, as well as for mass spectrometry analytical services of glycosylated proteins. Consequently, it will significantly aid in the discovery of diagnostic and therapeutic glycosylation biomarkers related to various diseases and health conditions.

What is claimed is:

1. A method for quantitatively analyzing glycan isomers based on mass spectrometry data, comprising:
substituting, by means of computer simulation, an isotope in a structural isomer of a to-be-quantified glycan isomer with an isotope having a similar mass, to obtain a simulated glycan isomer with a changed chemical formula and mass; and
quantifying the simulated glycan isomer based on the mass spectrometry data, to obtain quantitative results of different structural isomers,
wherein a difference between a mass of the simulated glycan isomer and a mass of the to-be-quantified glycan isomer is less than or equal to 0.2 Da.

2. The method according to claim 1, wherein:
x is a serial number of respective structural isomers of the to-be-quantified glycan isomers sorted in an ascending order of glycan ID number, the serial number being a natural number from 1 to n; and
the computer simulation is performed based on the number of N and the number of O in a chemical formula of the to-be-quantified glycan isomer, the computer simulation comprising any one of the following steps:
A1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
A2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
A3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, until k 160 are removed; for the structural isomer with the serial number of x, removing m ¹⁴N from the chemical formula, adding m ¹³C and m ¹H in the chemical formula, and removing (x−m−1) 160 from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da;
wherein the to-be-quantified glycan isomers comprise n structural isomers, where n is a natural number; m is a natural number; and k is a natural number.

3. A method for quantitatively analyzing a glycopeptide containing glycan isomers in mass spectrometry data, comprising:
substituting, by means of computer simulation, an isotope in the glycan isomer contained in the glycopeptide with an isotope having a similar mass, to obtain a simulated glycan isomer with a changed chemical formula and mass, and to obtain a glycopeptide containing the simulated glycan isomer; and
quantifying, by means of a mass spectrometry data quantification software, the glycopeptide containing the simulated glycan isomer based on the mass spectrometry data, to obtain quantitative results of the glycopeptide containing the glycan isomers of different structures,
wherein a difference between a mass of the simulated glycan isomer and a mass of the glycan isomer is less than or equal to 0.2 Da.

4. The method according to claim 3, wherein:
x is a serial number of respective structural isomers of the to-be-quantified glycan isomers sorted in an ascending order of glycan ID number, the serial number being a natural number and ranging from 1 to n; and
the computer simulation is performed based on the number of N and the number of O in a chemical formula of the to-be-quantified glycan isomer, the computer simulation comprising any one of the following steps:
A1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
A2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
A3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, until k 160 are removed; for the structural isomer with the serial number of x, removing m ¹⁴N from the chemical formula, adding m ¹³C and m ¹H in the chemical formula, and removing (x−m−1) 160 from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da;
wherein the to-be-quantified glycan isomers comprise n structural isomers, where n is a natural number; m is a natural number; and k is a natural number.

5. The method according to claim 3, wherein the mass spectrometry data quantification software is Skyline software.

6. A device for quantitatively analyzing a glycopeptide containing glycan isomers in mass spectrometry data, the device comprising:
B1) mass spectrometry data acquisition module configured to acquire mass spectrometry data of a sample;
B2) glycopeptide identification module configured to identify a glycopeptide contained in the sample based on the mass spectrometry data; and
B3) glycopeptide quantification module configured to quantify the glycopeptide, the glycopeptide quantification module comprising:
B3-1) glycan isomer simulation module configured to obtain a simulated glycan isomer and a glycopeptide containing the simulated glycan isomer through computer simulation of the glycan isomers having different structures contained in the glycopeptide; and
B3-2) glycopeptide quantification module configured to quantify, by means of a mass spectrometry data quantification software, the glycopeptide containing the simulated glycan isomer, to obtain quantitative results of the glycopeptide containing the glycan isomer.

7. The device according to claim 6, wherein:
x is a serial number of respective structural isomers of the to-be-quantified glycan isomers sorted in an ascending order of glycan ID number, the serial number being a natural number and ranging from 1 to n; and
the computer simulation is performed based on the number of N and the number of O in a chemical formula of the to-be-quantified glycan isomer, the computer simulation comprises any one of the following steps:
C1) when the number m of N in the chemical formula is greater than or equal to the number of the structural isomers of the glycan isomers minus 1, for a structural isomer with the serial number of x, removing (x−1) ¹⁴N from the chemical formula, and adding (x−1) ¹³C and (x−1) ¹H in the chemical formula, to obtain the simulated glycan isomer, wherein compared with the to-be-quantified glycan isomer, a mass of the simulated glycan isomer is increased by (x−1)×0.008106 Da;
C2) when the number m of N in the chemical formula is smaller than the number of the structural isomers of the glycan isomers minus 1, and when a sum (m+k) of the number m of N and the number k of O is greater than or equal to the number of the structural isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H, to obtain the simulated glycan isomer, wherein compared with the to-be-quantified glycan isomer, the mass of the simulated glycan isomer is increased by (x−1)×0.008106 Da or m×0.008106 Da+(x−m−1)×0.013019 Da; and
C3) when the sum (m+k) of the number m of N and the number k of O in the chemical formula is smaller than the number of the structural isomers of the glycan isomers minus 1, for the structural isomer with the serial number of x, removing (x−1) ¹⁴N from the chemical formula, and adding (x−1) ¹³C and (x−1) ¹H in the chemical formula, until m ¹⁴N are removed; for the structural isomer with the serial number of x, removing m ¹⁴N from the chemical formula, adding m ¹³C and m ¹H in the chemical formula, and removing (x−m−1) ¹⁶O from the chemical formula, and adding (x−m−1) ¹⁵N and (x−m−1) ¹H in the chemical formula, until k 160 are removed; then for the structural isomer with the serial number of x, removing m ¹⁴N from the chemical formula, adding m ¹³C and m ¹H in the chemical formula, and removing (x−m−1) ¹⁶O from the chemical formula, adding (x−m−1) ¹⁵N and (x−m−1) ¹H in the chemical formula, removing (x−m−k−1) ¹²C and (x−m−k−1) ¹H from the chemical formula, and adding (x−m−k−1) ¹⁵N in the chemical formula, to obtain the simulated glycan isomer, wherein compared with the to-be-quantified glycan isomer, the mass of the simulated glycan isomer is increased by (x−1)×0.008106 Da, or m×0.008106 Da+(x−m−1)×0.013019 Da, or m×0.008106 Da+k×0.013019 Da+(x−m−k−1)×0.0233 Da;
wherein the to-be-quantified glycan isomers comprise n structural isomers, where n is a natural number; m is a natural number; and k is a natural number.

8. The device according to claim 6, wherein the mass spectrometry data quantification software is Skyline software.

9. A non-transitory computer-readable storage medium, having a computer program stored thereon, wherein the computer program enables a computer to perform steps of the method according to claim 1.

10. A non-transitory computer-readable storage medium, having a computer program stored thereon, wherein the computer program enables a computer to perform steps of the method according to claim 2.

11. A non-transitory computer-readable storage medium, having a computer program stored thereon, wherein the computer program enables a computer to perform steps of the method according to claim 3.

12. A non-transitory computer-readable storage medium, having a computer program stored thereon, wherein the computer program enables a computer to perform steps of the method according to claim 4.

13. A non-transitory computer-readable storage medium, having a computer program stored thereon, wherein the computer program enables a computer to perform steps of the method according to claim 5.
